(19) United States
(12) Patent Application Publication
(10) Pub. No.: US 2021/0059984 A1
(43) Pub. Date: Mar. 4, 2021

(54) METHOD AND COMPOSITION FOR REVERSING COGNITIVE IMPAIRMENTS AND LOSS USING SGLT2 INHIBITORS

(71) Applicant: TAISHO PHARMACEUTICAL CO., LTD., Tokyo (JP)

(72) Inventors: Fan FAN, Jackson, MS (US); Richard J. ROMAN, Jackson, MS (US); Shaoxun WANG, Jackson, MS (US)

(73) Assignee: TAISHO PHARMACEUTICAL CO., LTD., Tokyo (JP)

(21) Appl. No.: 16/556,948

(22) Filed: Aug. 30, 2019

(51) Int. Cl. A61K 31/382; A61P 25/28; A61P 3/06

(52) U.S. Cl. CPC A61K 31/382; A61P 3/06; A61P 25/28

(57) ABSTRACT

A new drug useful in the treatment of cognitive impairment is provided, in which an effective amount of luseogliflozin or a pharmaceutically acceptable salt thereof, or a hydrate of luseogliflozin or the salt is contained as an active ingredient.

### TECHNICAL FIELD

The present invention relates to a method and composition for reversing cognitive impairments and loss using SGLT2 Inhibitors.

### BACKGROUND ART

With the recent rapid increase in the elderly population, the number of patients with cognitive impairments has also increased.

While cognitive impairments is classified into several types, the majority consists of dementias of Alzheimer type, cerebrovascular dementias, and dementias with Lewy bodies. A current understanding goes that in dementias of Alzheimer type, unique proteins called β-amyloid and tau accumulate in the brain, leading to necrosis and decrease of nerve cells, which eventually results in cognitive dysfunction, and that in cerebrovascular dementias, such diseases as cerebral infarction, cerebral hemorrhage, and subarachnoid hemorrhage cause necrosis of brain cells, which eventually results in cognitive dysfunction. Vascular lesions are caused by arterial sclerosis and risk factors of arterial sclerosis include hypertension, diabetes mellitus, cardiac diseases, lipid abnormalities, smoking, and so forth; cerebrovascular dementias may well be considered to be associated with life style.

Cognitive impairments is appropriately taken care of by such methods as medication therapy and rehabilitation but no methods are currently available to cure cognitive impairments completely. As medication therapies, donepezil hydrochloride, galantamine, rivastigmine, memantine, etc. are used against dementias of Alzheimer type, with donepezil hydrochloride being also used against dementias with Lewy bodies. For treatment of cerebrovascular dementias, cilostazol, nicergoline, etc. are employed. Use of these drugs is effective for delaying the progress of cognitive impairments by a certain degree or ameliorating its symptoms; however, their efficacy is still insufficient and the development of effective therapeutics for cognitive impairments is a global need.

Diabetes mellitus (DM) is considered as one of leading risk factors for aging-related dementia, including Alzheimer's disease and vascular cognitive impairment, but the contribution of chronic diabetes to dementia is not fully elucidated. It is believed that diabetes promotes endothelial dysfunction and enhances reactive oxygen species (ROS) production, which may increase the susceptibility to brain ischemic injury. However, the evidence is still unclear whether localized hypoxia induces dementia in diabetic subjects alone or if DM also impairs cerebral blood flow (CBF) autoregulation and this contributes to dementia.

Clinicians have now identified of new state of Diabetes mellitus, referred to as Type 3 Diabetes, where patient exhibit peripheral and central nervous systems damage caused by persistent hyperglycemia. Subjects with chronic diabetes often exhibit cerebral vascular dysfunction, blood-brain barrier leakage, and inflammation that may contribute to the development of neurodegeneration and eventually dementia.

Cognitive impairment and dementia in elderly patients with long-standing or chronic diabetes is an emerging global medical crisis. Alzheimer's disease accounts for 60-80% of dementia cases. Alzheimer's disease and diabetes mellitus are two of the most common diseases of aging. An estimated 5.4 million of Americans, 1 in 8 people age ≥65 and 1 in 2 people age ≥85 are affected by Alzheimer's disease. The incidence of diabetes is 26.9% in patients of ≥65 years of age in the US, with 29 million of Americans having this disease. The Medicare cost for the treatment of dementia and Alzheimer's disease is $159 billion annually and is projected to rise to $511 billion by 2040. Diabetes and pre-diabetes cost America $322 billion per year. The inter-relationship of the two diseases have been speculated but remains unknown. There is an urgent need to understand the mechanisms for the development of new therapeutic strategies to delay the onset and progression of these devastating diseases.

With Diabetes mellitus's association with cognitive impairments being also recognized, rosiglitazone maleate having a potent insulin enhancing action has been developed as an anti-Alzheimer's disease agent (Non-Patent Document 1). Inhibitors of sodium-glucose cotransporter 2 (hereinafter sometimes designated as SGLT2) are therapeutics for diabetes mellitus, which bring blood glucose levels to normal by inhibiting the glycemic reabsorption from proximal tubules in the kidneys so as to promote urinary glucose excretion. SGLT2 inhibitors are therapeutics that present less side effects and which have a different mechanism of action than drugs that act on insulin secretion.

It has been reported that certain types of SGLT2 inhibitors suggest efficacy for improving cognitive functions. Non-Patent Document 2 shows the cognitive function improving effect of empagliflozin in diabetic mice; Non-Patent Document 3 describes the relation between the treatments of diabetes mellitus and Alzheimer's disease based on the action of canaglifolzin for inhibiting acetylcholinesterase and SGLT2; Non-Patent Document 4 describes a study in which following 12-month administration of canaglifolzin, empagliflozin, dapagliflozin, and incretins to diabetic patients (with a mean age of 77 years), their cognitive performance, the results of biochemical tests, etc. were evaluated, with the result of no worsening of their cognitive functions, which led to the estimation that normal blood glucose levels will prevent or improve cognitive functions and that the SGLT2 inhibitors and incretins will play a role in preventing cognitive impairment. Another report, refers to the utility of SGLT2 inhibitors as drugs for removing aged cells, while listing various aging-related diseases including anti-Alzheimer's disease and dementia (Patent Document 1).

However, no reports have been published demonstrating the cognitive function improving effect of luseogliflozin. What is more, there is no report that demonstrates an improvement of cognitive functions by SGLT2 inhibitors applied to diabetic animals that are many weeks old, nor is there a report that demonstrates the action of SGLT2 inhibitors in cerebral vessels.

### CITATION LIST

### Patent Literature

Patent Document: WO2018/043463, patent application filed by Niigata University disclosing an invention entitled “Drug for Removing Aged Cells”

### Non-Patent Literature

Non-Patent Document 1: Rosiglitazone Monotherapy in Mild-to-Moderate Alzheimer's Disease: Results from a Randomized, Double-Blind, Placebo-Controlled Phase III Study (Dement Geriatr Cogn Disord 2010; 30: 131-146)
Non-Patent Document 2: Glycemic control with empagliflozin, a novel selective SGLT2 inhibitor, ameliorates cerebrovascular injury and cognitive dysfunction in obese and type 2 diabetic mice (Cardiovasc Diabetol. 2014; 13: 148)
Non-Patent Document 3: Invokana (Canagliflozin) as a dual inhibitor of acetylcholinesterase and sodium glucose co-transporter 2: advancement in Alzheimer's disease-diabetes type 2 linkage via an enzoinformatics study (CNS Neurol Disord Drug Targets. 2014 April; 13 (3): 447-51)
Non-Patent Document 4: 12-month effects of incretins versus SGLT2-Inhibitors on cognitive performance and metabolic profile. A randomized clinical trial in the elderly with Type-2 diabetes mellitus (Clin Pharmacol. 2018 Oct. 9; 10: 141-151. doi: 10.2147/CPAA.S164785. eCollection 2018)

### SUMMARY OF INVENTION

### Technical Problem

The present invention has as its objective providing anew drug having a cognitive function improving effect or a cognitive dysfunction suppressing effect.

### Solution to Problem

The present inventors made an intensive study in order to solve the above-mentioned problem and focusing on the involvement of a hyperglycemic condition in cognitive function, eventually found a composition and method for reversing the loss of cognitive function in animals having prolonged hyperglycemia, Type 3 Diabetes, using SGLT2 Inhibitors that provide for the treatment and reversal of cerebral vascular function, in particular, that luseogliflozin has the following effects in T2DN rats as a diabetic animal model:

(1) Restoring myogenic response in the middle cerebral artery, and autoregulation of cerebral blood flow;
(2) Restoring stimulatory response of cerebral vessels;
(3) Restoring blood-brain barrier disorder.

Based on the finding that luseogliflozin represented by the following formula improves cognitive functions in T2DN rats, the present inventors realized that it would be useful in the prevention and/or treatment of cognitive impairments, which eventually led to the accomplishment of the present invention.

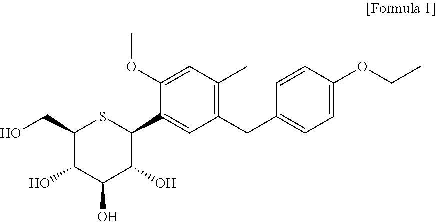 [SMILES: CCOc1ccc(Cc2cc([C@@H]3S[C@H](CO)[C@@H](O)[C@H](O)[C@H]3O)c(OC)cc2C)cc1]

Briefly, the present invention provides:

(1) A method of treating cognitive impairments which comprises administering to a subject in need thereof an effective amount of luseogliflozin or a pharmaceutically acceptable salt thereof, or a hydrate of luseogliflozin or the salt;
(2) The method as described in (1), wherein the cognitive impairments is diabetic cognitive impairments;
(3) A method of treating a cerebrovascular dementias which comprises to a subject in need thereof an effective amount of luseogliflozin or a pharmaceutically acceptable salt thereof, or a hydrate of luseogliflozin or the salt.

### Advantageous Effects of Invention

Luseogliflozin is capable of improving cognitive functions and if it is used in the prevention or treatment of cognitive impairments, patients can maintain the QOL of their live a life for an extended period of time. At the same time, luseogliflozin, having a blood glucose lowering effect, can contribute to the prevention or treatment of various diseases attributed to hyperglycemia (this benefit is absent from conventional drugs for treating and/or preventing cognitive impairments) and hence is promising as a drug having new actions; as a further advantage, if luseogliflozin is used in combination with exercise or dietary therapy, the therapeutic effect would be enhanced, whereby a new therapeutic method can be provided.

### BRIEF DESCRIPTION OF DRAWINGS

The patent or application f ile contains at least one drawing executed in color. Copies of this patent or patent application publication with color drawing(s) will be provided by the United States Patent and Trademark Of f ice upon request and payment of the necessary f ee.

FIG. 1 Elderly T2DN rats exhibit impaired pressure-induced myogenic response in isolated MCA.

FIG. 2. Autoregulation of Cerebral blood flow in response to elevations in middle cerebral artery pressure (MAP) is impaired in T2DN and type 1 diabetic STZ treated SD rats relative to nondiabetic SD rats

FIG. 3. Plasma glucose and Hb_{A1C} levels are reduced in 18-month diabetic T2DN rats treated luseogliflozin.

FIG. 4. The myogenic response of middle cerebral artery is restored by lowering plasma glucose levels in luseogliflozin treated old diabetic T2DN rats.

FIG. 5. Autoregulation of cerebral blood flow is restored in luseogliflozin treated aged diabetic T2DN rats.

FIG. 6. Evan's blue is elevated in T2DN rats showing blood brain leakage that rescued by administration of Luseogliflozin to old diabetic T2DN rats.

FIG. 7. Functional hyperemia in response to whisker stimulation is restored in Luseogliflozin treated old diabetic T2DN rats.

FIG. 8. Cognitive impairments in 8-arm water maze test are rescued in Luseogliflozin treated old diabetic T2DN rats.

FIG. 9. (A) The brain expression of Aβ, p-Tau, IL-1β and GFAP detected using Western blot and (B) expression of Tau examined by immunostaining in 12-18 months T2DN and SD rats.

### DESCRIPTION OF EMBODIMENTS

While the terms used herein are believed to be well understood by one of ordinary disclosed subject matter.

The term “Diabetes mellitus” is a metabolic syndrome in which impaired secretion of insulin or a dysfunction in the insulin response of a target organ causes glucose metabolism abnormalities which in turn results in chronic hyperglycemia.

The term “persistent or chronic hyperglycemia” as used herein means either one of the following conditions: (1) the fasting blood glucose level is 126 mg/dL or more; (2) the 2-hr value is 200 mg/dL or more in a 75 g oral glucose tolerance test (OGTT); (3) the non-fasting blood glucose level is 200 mg/dL or more; (4) the value of HbA_{1c} is 6.5% or more. Diabetes mellitus covers type 1 diabetes, type 2 diabetes, and other types of diabetes caused by specific reasons. A preclinical stage of diabetes is called impaired glucose tolerance (IGT) in which HbA_{1c} is less than 6.5% but either one of the following conditions is satisfied: (1) the fasting blood glucose level is 110-125 mg/dL; (2) the blood glucose level two hours after 75 g glucose load is 140-199 mg/dL.

The term “an elderly subject” as used herein refers to in particular, age 50 years or older, more specifically, age 70 years or older.

The term “cerebrovascular impaired diabetics” refers to elderly subjects that have persistent hyperglycemia for at least one month to one year or longer and exhibit cerebral vascular dysfunction, blood-brain barrier leakage, or inflammation.

The term “cognitive impairment” refers the loss of cognitive function in elderly subjects due to cerebral vascular dysfunction, blood-brain barrier leakage, or inflammation.

The term “cerebral blood flow dysfunction” refers to changes in blood flow caused by cerebrovascular lesions including brain infarction and hemorrhage, as well as a blood flow dysfunction caused by hardening of brain vessels. Cerebrovascular dementia is a form of dementia that manifests upon partial necrosis of brain cells due to cerebral blood dysfunction.

The term “treatment” or “treating” as used herein covers not only the improvement of cognitive function, but also the reversing the loss of cognitive function caused by persistent or chronic hyperglycemia. In addition, it covers all practices of medical therapy associated with cognitive dysfunction and more advanced states in persistent or chronic hyperglycemic aged patients, who are suffering from impaired cognitive function, including dose reduction of therapeutics for dementia, suppression and stabilization of various symptoms due to cognitive dysfunction, suppression and delaying of the onset and progression of dementia, reduction of side effects from therapeutics for dementia, and survival prolongation of patients with dementia.

The term “long-term administration” refers the continuous treatment of a hyperglycemic elderly subject for at least one week, one month, one year or longer until the cognitive impairments are improved or reversed in a cerebrovascular impaired diabetic subject.

The term “long-term maintenance” refers to continuous treatment of a treated cerebrovascular impaired diabetic subject after their cognitive impairments are improved or reversed.

The therapeutic drug of the present invention can usually be administered systemically or topically in an oral or parenteral dosage form and can be formulated as an appropriate preparation that suits the intended route of administration. The preparation may be processed by conventional means into oral drugs such as tablets, granules, powders, capsules, emulsions, suspensions and syrups, or injections such as sterile solutions or in suspension form, as well as drugs for cutaneous application including lotions, creams, ointments, and patches. Preferred dosage forms are oral drugs such as tablets, granules, powders, capsules, emulsions, suspensions and syrups.

Sodium-glucose cotransporter-2 (SGLT2) proteins are expressed in the proximal convoluted tubule of the kidneys. These transporters are an ideal target for the treatment of diabetes because they are responsible for roughly 90% of filtered glucose reabsorption. The normal renal threshold for reabsorption of glucose corresponds to a serum glucose concentration of 180 mg/dL. In patients with type 2 diabetes, this threshold can increase and the expression of the SGLT2 can be up-regulated causing a maladaptive response that worsens hyperglycemia. Selective inhibition of SGLT2 inhibitors can reduce this threshold to as low as 40 to 120 mg/dL. Recently, SGLT2 inhibitors have moderate benefits on atherosclerotic major adverse cardiovascular events that appear confined to patients with established atherosclerotic cardiovascular disease. Currently there are three SGLT2 selective inhibitors approved by the Food and Drug Administration (FDA), for the treatment of type II diabetes which include are mono, dual, and triple therapy: canagliflozin (Invokana®), dapagliflozin (Farxiga®) and empagliflozin (Jardiance®). There are several other similar compounds in the pipeline or in development that may be approved in the near future that include ipragliflozin, luseogliflozin, tofogliflozin, ertugliflozin, LX4211, and EGT0001442. Of the three FDA approved drugs, empagliflozin has the greatest selectivity for SGLT2 compared to SGLT1, while canagliflozin is the least selective. Further, four combination drugs have also been approved by the FDA: canagliflozin/metformin (Invokamet®), dapagliflozin/metformin (Xigduo XR®), empagliflozin/metformin (Synjardy®) and empagliflozin/linagliptin (Glyxambi®).

The dose of the therapeutic SGLT2 inhibitor drug according to the present invention varies with such factors as age, body weight, symptoms, therapeutic efficacy, method of administration, and time of application and depends on various conditions. As one embodiment, that is not meant to be limiting, the dose of SGLT2 inhibitor drug varies with amount administered and how it is administered. For example, a daily dose of luseogliflozin is administered orally, preferably 0.1-50 mg, more preferably 0.5-5 mg, even more preferably 0.5-2.5 mg, per 60 kg patient.

The present invention has also other embodiments as follows.

In the first embodiment, a method for the administration of an effect amount of an antidiabetic drug into an elderly type 2 diabetic animal with persistent or chronic hyperglycemia that resolves the levels of plasma glucose and HbA1c to near normal. A further embodiment is that animals have persistent or chronic hyperglycemia for a period at least one month to one year or longer. A preferred embodiment is a validated glycemic control drug that is found in renal proximal tubule and reduce renal glucose reabsorption while primarily promoting urinary glucose excretion. This compound does not cross the blood-brain barrier with molecular weight 434.5, where the cutoff is 400 (William M Pardridge, Journal of Cerebral Blood Flow & Metabolism. 2012).

Sodium-glucose cotransporter-2 (SGLT2) proteins are mainly expressed in the proximal convoluted tubule of the kidneys and are an ideal target for the treatment of diabetes because they are responsible for roughly 90% of filtered glucose reabsorption. A still further embodiment are classes of drugs that control hyperglycemia by acting at (SGLT2) proteins by inhibiting the function, known as SGLT2 inhibitors. Non-limiting examples of SGLT2 inhibitors approved by the FDA for type II diabetes include canagliflozin (Invokana®), dapagliflozin (Farxiga®) and empagliflozin (Jardiance®). There are several other similar compounds in the pipeline that may be approved in the near future, one of which is Luseogliflozin. Of the three FDA approved drugs, empagliflozin has the greatest selectivity for SGLT2 compared to SGLT1, while canagliflozin is the least selective.

In a second embodiment, a method for the administration of an effect amount of an antidiabetic SGLT2 inhibitor drug into an elderly type 2 chronic hyperglycemia animal having one or more of the following symptoms including cerebral vascular dysfunction, blood-brain barrier leakage, or inflammation that improves, rescues, or reverses one or more symptoms. The manifestations are known to be caused by impaired cerebral blood flow autoregulation and the myogenic response of the cerebral arterioles. In a further embodiment, the administration is a long-term continuous administration of an effect amount of an antidiabetic SGLT2 inhibitor drug into an elderly type 2 chronic hyperglycemia animal for at least one week, one month, one year, or longer that improves, rescues, or reverses one of more symptoms of cerebral vascular dysfunction, blood-brain barrier leakage, or inflammation. The administration of the therapeutic SGLT2 inhibitor drug according to the dose recommended to control to the levels of plasma glucose and HbA1c to near normal, which varies with such factors as age, body weight, symptoms, therapeutic efficacy, method of administration, and time of application and depends on various conditions. As an additional embodiment, that is not meant to be limiting, the dose of SGLT2 inhibitor drug varies with amount administered and how it is administered, a daily dose of luseogliflozin is administered orally, preferably 0.1-50 mg, more preferably 0.5-5 mg, even more preferably 0.5-2.5 mg, per 60 kg patient.

In a third embodiment, a method for the continuous administration of an effective amount of an antidiabetic SGLT2 inhibitor drug into an elderly type 2 animal to improve, rescue, or reverse cognitive impairments due to cerebral vascular dysfunction due caused by chronic hyperglycemia. In a further embodiment, the administration of an effect amount of an antidiabetic SGLT2 inhibitor drug is administered until the cognitive impairments are improved or reversed in a cerebrovascular impaired diabetic subject.

In the fourth embodiment, a method for the continuous administration of an effect amount of an antidiabetic SGLT2 inhibitor drug into an elderly type 2 animal to maintain the improvement or reversal cognitive impairments and cerebral vascular dysfunction due caused by chronic hyperglycemia.

In the fifth embodiment, a method for the continuous administration of an effect amount of an antidiabetic SGLT2 inhibitor drug into an elderly type 2 animal to maintain the improvement or reversal of cognitive impairments and cerebral vascular dysfunction is co-administered with other classes of antidiabetic drugs or other SGLT2 inhibitors. As an additional embodiment, that is not meant to be limiting, the dose of SGLT2 inhibitor drug varies with amount administered and how it is administered with antidiabetic, such as the four combination drugs also been approved by the FDA that canagliflozin/metformin (Invokamet®), dapagliflozin/metformin (Xigduo XR®), empagliflozin/metformin (Synjardy®) and empagliflozin/linagliptin (Glyxambi®). In still other embodiments, SGLT2 inhibitor drug is co-administered with other classes of ACE inhibitors or angiotensin type I receptor blockers affecting cerebrovascular function to provide further improvements.

In a sixth embodiment, a method for the continuous administration of an effective amount of an antidiabetic SGLT2 inhibitor drug into an elderly type 2 animal to improve, rescue, or reverse cognitive deficits associated with elevations in Aβ protein and Tau resulting in Alzheimer's disease.

### EXAMPLES

The present invention will now be described in greater detail by means of working examples and other data, but it should be understood that the present invention is by no means limited to those working examples and other data.

### Example 1: Type I and Type II Diabetic Rats Exhibiting Persistent Hyperglycemia

Cerebral blood flow autoregulation is a critical homeostatic mechanism that protects the brain from vascular damage and cerebral edema following systemic hypertension and from ischemic injury following embolization or hypotension. Autoregulation of cerebral blood flow in response to elevations in pressure is primarily mediated by the myogenic response, which is an intrinsic property of vascular smooth muscle cells to constrict in response to elevations in transmural pressure. The myogenic response and autoregulation in the brain are impaired in aging or diabetes, and this cerebral vascular dysfunction is further exacerbated in aging combining with diabetes.

In the invention, it was determined whether this comorbidity is solely due to persistent hyperglycemia and cerebral vascular dysfunction, or there is a synergistic effect of vascular and metabolic dysfunctions that promotes brain damage and accelerates cognitive deficits in aging diabetes and, in some cases, leads to Alzheimer's disease.

To study this correlation, unique type I and type II diabetic rat models were developed that have impaired autoregulation of cerebral blood flow. Surprisingly, it was found that these rats develop cognitive deficits associated with elevations in Aβ protein and Tau protein in the brain, the accepted characteristic biomarkers of Alzheimer's Disease.

Experiments were performed on 3- and 12-month male T2DN and SD rats and 9- and 30-week male Streptozotocin (STZ) treated type I diabetic SD rats. T2DN rats were prepared according to Diabetes, vol. 53, March 2004, pages 735-742. T2DN rats became diabetic at 3 months of age and first show signs of diabetic nephropathy by 6-months of age. Thus, 12 month old T2DN rats were exposed to chronic diabetes. Aged T2DN animals are defined as >half normal lifespan, since the mean lifespan of rats is around 18 months. In this study, 12-month T2DN rats were considered as aged or elderly subjects.

Measurements of arterial pressure and cortical blood flow (CBF) were performed by inserting cannulas in the femoral artery and vein for drug delivery and monitoring the mean arterial pressure (MAP). A closed cranial window was prepared on both sides of the brain by thinning the bone using a dental drill until the vessels on the surface of the brain could be visualized. Cortical blood flow (CBF) was measured using a laser Doppler flowmeter (LDF).

To measure the myogenic response of cerebral arteries, a section of the brain containing middle cerebral artery (MCA) was dissected and immediately placed in Tyrode's solution. A branch of the MCA was microdissected and cannulated with glass pipettes in a pressure myograph chamber filled with a physiologic salt solution (PSS). The vessels were equilibrated at a transmural pressure of 40 mmHg to develop spontaneous tone. After preconditioning, the inner diameter (ID) of MCA in response to intraluminal pressures ranging from 40 to 180 mmHg in steps of 20 mmHg was measured to access the myogenic response. Then, a passive pressure-diameter relationship was determined by replacing the bath solution with calcium-free PSS. The myogenic reactivity in response to an elevation in perfusion pressure was calculated by the formula: IDₚᵣₑₛₛᵤᵣₑ/D_{40 mmHg}×100(%).

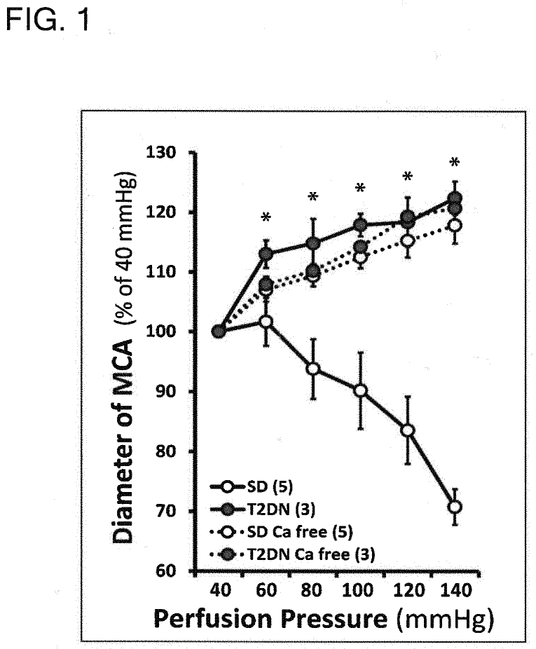

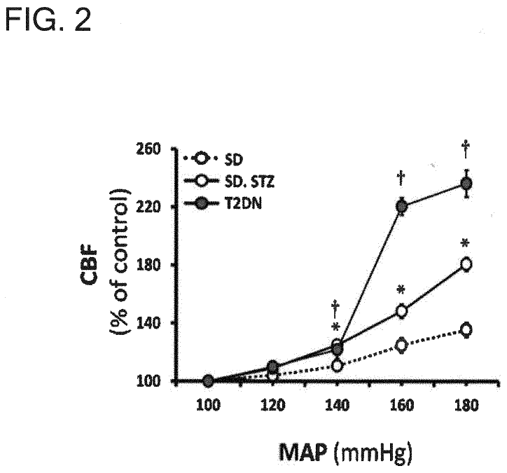

Elderly type II diabetic, T2DN, rats were found to have impaired myogenic response (FIG. 1). The diameter of the MCA was significantly increased as the pressure was increased in T2DN regardless of the calcium environment. In contrast, the MCA of elderly type 1 SD rats constricted in response to an elevation in transmural pressure and dilated passively after removal of calcium from the bath. T2DN and STZ-treated SD rats with type 1 diabetes showed significantly increased cerebral blood flow as MAP increased compared to control SD rats (FIG. 2). FIG. 2 also shows that CBF autoregulation was impaired to a greater extent in T2DN rats than in STZ treated type I diabetic SD rats.

### Example 2: The Effect of Treatment of T2DN Rats with SGLT2 Inhibitor on the Impaired Myogenic Response and Autoregulation of Cerebral Blood Flow

Unless otherwise indicated, all experiments using SGLT2 inhibitors were performed on 18-month old male Sprague Dawley (SD) and 14-month old T2DN rats, a genetic rat model of type II diabetes. Outbred SD rats purchased from Charles River were used as a nondiabetic control rat since identical genotypes are not necessary for this study. T2DN rats were prepared according to Diabetes, vol. 53, March 2004, pages 735-742. While SGLT2 is highly expressed in renal proximal tubule, an SGLT2 inhibitor does not have any known direct effect on the brain and is a validated glycemic control drug in diabetes. Treatment with an SGLT2 inhibitor began at 14-months of age and lasted for 4 months to determine whether control of hyperglycemia can slow the progression of cognitive dysfunction. Rats received either a vehicle or the SGLT2 inhibitor, luseogliflozin, at a dose of 20 mg/kg/day via drinking water. The SD rats were used as control.

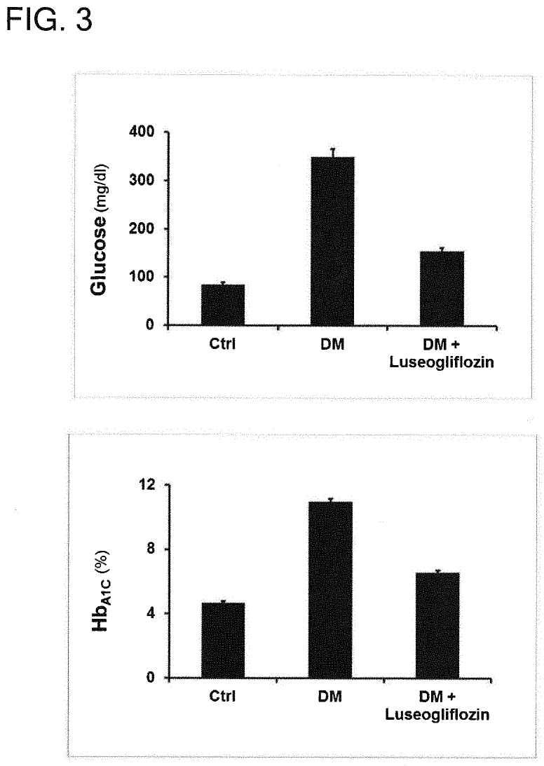

The levels of plasma glucose and HbA1c were measured by drawing a drop of blood from the tail vein during drug treatment and using traditional analytical methods to determine the concentration of plasma glucose and HbA1c. As shown in FIG. 3, the levels of glucose and HbA1c were substantially increased in untreated elderly diabetic rats. However, treatment with the SGLT2 inhibitor, luseogliflozin, reduced both the levels of plasma glucose and HbA1c to near normal. These results demonstrated that luseogliflozin was effective in normalizing plasma glucose and HbA1c.

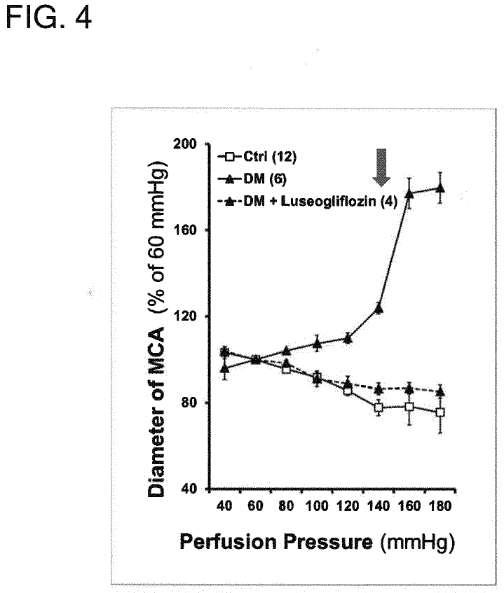

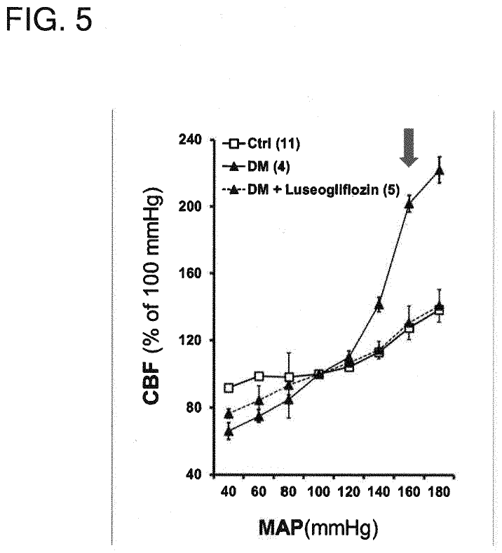

Next the effect of SGLT2 inhibitor was examined in the treatment of impaired myogenic response and autoregulation of cerebral blood flow was determined. In the figures below, Ctrl is control and DM is the aged diabetic T2DN rat. In FIG. 4, the administration of luseogliflozin completely reversed the increase in the diameters of middle cerebral artery upon exposure to higher perfusion pressures. The myogenic reactivity in response to an elevation in perfusion pressure was calculated by the formula: Dₚᵣₑₛₛᵤᵣₑ/ID_{60 mmHg}×100(%). Similarly, the substantial increase in the cerebral blood flow of untreated rat was restored to normal levels upon luseogliflozin treatment (FIG. 5). MAP was adjusted to 100 mmHg by i.v. administration of pentobarbital. After a 10-min equilibration period, baseline CBF was measured at 100 mmHg. MAP was then increased up to 180 mmHg in steps of 20 mmHg by infusing phenylephrine via the femoral vein. CBF was measured at each step when the MAP reached a steady state. The infusion of phenylephrine was then stopped which allowed MAP to return to 100 mmHg, and CBF was re-recorded. MAP was then reduced to 40 mmHg in steps of 20 mmHg by graded hemorrhage. CBF was obtained at each step when MAP achieved a steady state. Percentage change of CBF was calculated by the formula: [(CBF_{MAP}−CBF_{baseline})/CBF_{baseline}]×100(%). Together, the results demonstrated that the SGLT2 inhibitor was highly effective in restoring the myogenic response of cerebral arteries and correcting the cerebral blood flow impairments in type 2 diabetic animals.

To determine the impact of higher cerebral blood flow with extended middle cerebral artery diameters on the brain, the brains of SGLT2-treated and untreated rats were assessed for blood brain leakage (BBB). Evans blue (EB) dye was used to compare the degree of BBB leakage. EB was injected via the femoral vein, and MAP was maintained at 180 mmHg for 1 hour by adjusting the infusion rate of phenylephrine. After the equilibration period, the brain was subjected to intracardiac perfusion with saline to flush EB out of the vascular lumen and, thereafter, a brain image was obtained. The brain was then homogenized and BBB leakage was quantitated using a fluorescent microplate reader at excitation and emission wavelengths of 620 nm and 680 nm.

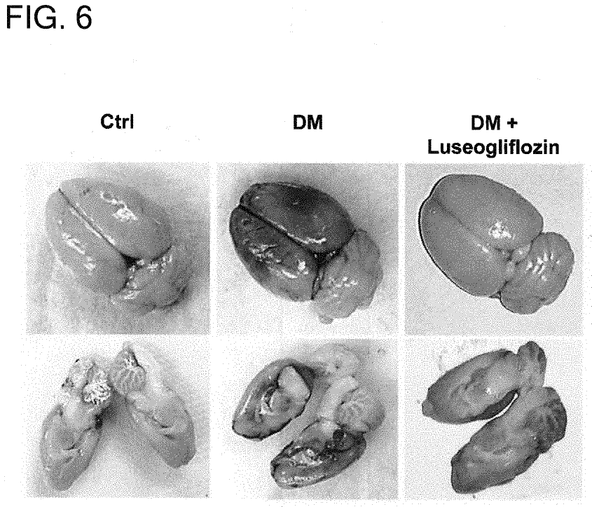

FIG. 6 dramatically depicts the effect of SGLT2 inhibitor in reversing the long-term impact of cerebral blood flow damage on BBB leakage in aged type 2 rats. The blood brain leakage is demonstrated by the degree of blue staining in the brain tissues. Note that long-term Luseogliflozin treatment completely reversed the blood brain barrier damage observed in T2DN rats.

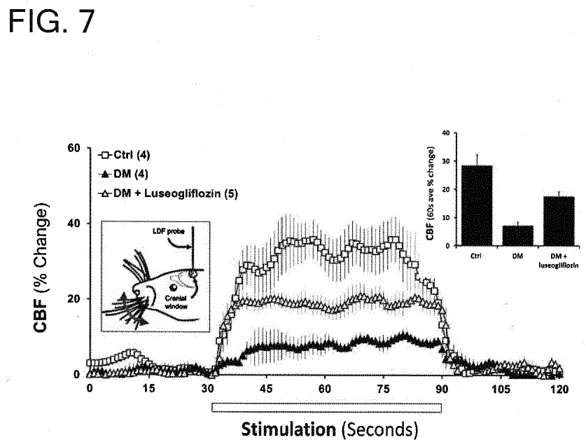

Elderly T2DN rats also exhibit impaired neurovascular coupling and a functional hyperemic response to whisker stimulation that is closely aligned with cognitive dysfunction (see insert of FIG. 7). In these studies, neurovascular coupling (NVC) responses were investigated prior to cerebral autoregulation study. The whisker-barrel cortex was activated by whisker stimulation (10 Hz for 60 seconds) on the right side through a mesh connected to a custom-made stimulation device (Am J Physiol Heart Circ Physiol. 2008 August; 295(2): H619-H631). Cortical blood flow (CBF) was measured using a laser Doppler flowmeter (LDF). LDF probe was placed above somatosensory cortex for the recording of LDF signal change (CBFchange). Percentage changes in CBF at various perfusion pressures vs. CBF at perfusion pressure of 100 mmHg were determined in response to whisker stimulation.

In the upper right panel of FIG. 7, the cerebral blood flow is shown for control, untreated chronic type 2 diabetic and SGLT2 inhibitor treated animals. A comparison of the recovery of functional response in stimulation in old diabetic T2DN (DM) animals showed a striking improvement and return to a normal response.

### Example 3: T2DN Animals Treated with SGLT2 Inhibitor which Rescues Cognitive Impairments and Loss

These results above are very consistent in demonstrating that SGLT2 inhibitors restore cerebral blood flow while reversing metrics corresponding to neurological damage of brain tissue. However, it is unknown whether their rescue can reverse the damage to cognitive function losses due to persistent exposure to increased cerebral blood flow and associated features.

The cognitive function of chronic diabetic animals were tested in terms of spatial learning and memory using an eight-arm radial water maze test that does not require deprivation or extensive training. Eight-arm radial water maze test was performed using a procedure for testing spatial learning and short and long-term memory that does not require deprivation or extensive training. The rats are trained to identify a platform marked with a visual cue in one of the 8 arms of the pool to escape. Four memory trials are performed at 2, 24, 48 and 72 hrs after the training, which consist of 4 consecutive swims. The time to reach the platform (latency), the number of errors, distance traveled and swim speed were recorded (Nat Protoc 1: 1671-1679, 2006).

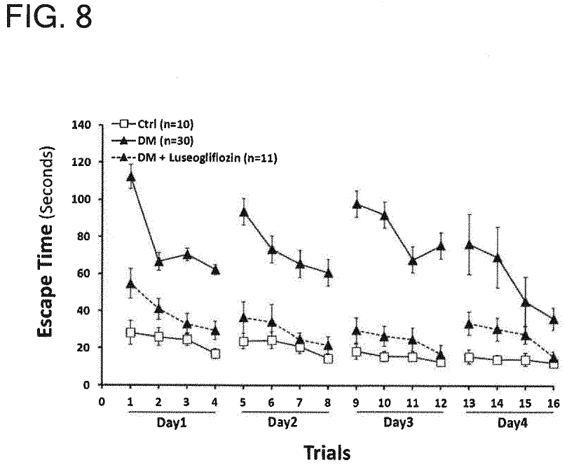

Control, DM and DM luseogliflozin treated rats were trained on the first day to recognize the platform located in one of the eight arms to escape, and repeated tests were performed 2, 24, 48, and 72 hours post training. The time taken by the rats to reach the platform was recorded. Animals that were administered luseogliflozin exhibited a dramatic improvement in cognitive performance as shown by the reduced escape time similar to normal rats demonstrated over the course of a 4 day trial (FIG. 8). This finding was completely unexpected and would not be expected if the loss of cognitive function was due to diabetes induced neurodegeneration. Further, these results suggest that the loss of cognitive function was related to vascular dysfunction in chronic subjects that can be reversed as shown in FIGS. 4 and 5 by correction of hyperglycemia with long-term Luseogliflozin administration. The marked reversal of functional cognitive loss using Luseogliflozin treatment in the chronic diabetic animals that already display cognitive dysfunction is shown for the first time.

### Example 4: Elderly Diabetic Animals Treated with SGLT2 Inhibitor to Reverse Cognitive Impairments Including Alzheimer's Disease

The underlying cause of neurodegenerative Alzheimer's Disease is focused on cholinergic deficiency, extracellular amyloid beta (Aβ) accumulation that forms plaques in the brain, and Tau protein abnormalities leading to neurofibrillary tangles. However, current treatments and clinical trials targeting these pathways such as using inhibitors of acetylcholinesterase, γ secretase or Tau have not been effective which has led the community to find alternatives. There is increased evidence indicating that cerebral vascular dysfunction plays an important role in the development of dementia. For example, buildup of Aβ protein leads to amyloid angiopathy that has been found in 90% of post-mortem brains collected from AD patients. This pathological observation is associated with cerebral vascular dysfunctions, including ruptures of BBB, microhemorrhages and inflammation. However, whether the vascular lesions are secondary to the accumulation of Aβ protein or are the initiating factors to increase Aβ production and/or decrease Aβ clearance is still unclear.

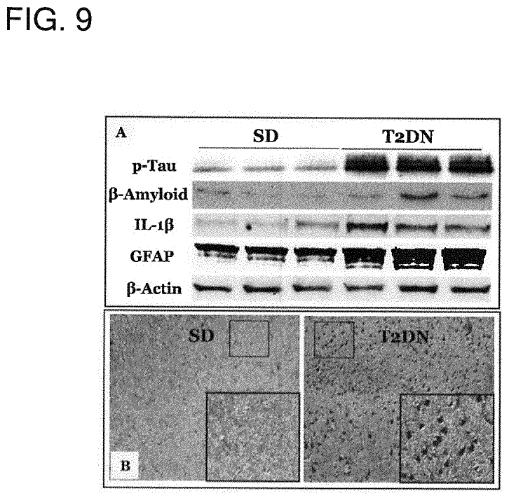

Persistent hyperglycemia, cerebral vascular dysfunction including impaired cerebral blood flow or a synergistic effect of vascular and metabolic dysfunction promotes brain damage and accelerates cognitive deficits in aging diabetes which could lead to Alzheimer's Disease. Supporting this possibility, the neurodegeneration in elderly chronic T2DN rats was associated with elevated expression of beta-amyloid and Tau protein in the brain (FIGS. 9A & B), suggesting these diabetic rats expressed the characteristic biomarkers of Alzheimer's Disease. The elevated expression of GFAP and IL-1β in aged T2DN rats suggests that glial activation and inflammation may link aging, diabetes and cognitive deficits (FIG. 9A). Beta-Actin was used as loading control.

Further, it is demonstrated that the abnormal cerebral vascular function in elderly hypertensive Dahl salt-sensitive (SS) rats was rescued when autoregulation of cerebral blood flow is restored in transgenic CYP4A1 rats on the SS genetic background. Preliminary analysis of related human genes (CYP4F2, CYP4A11) involved in autoregulation of cerebral blood flow (CBF) were associated with cognitive impairment in The Atherosclerosis Risk in Communities-Neurocognitive Study (ARIC-NCS) population of elderly diabetic patients having a higher incidence of Alzheimer's Disease.

The elderly chronic T2DN animals having cerebral vascular dysfunction are administered continuously with a SGLT2 inhibitor. The long term impact of cerebral vascular dysfunction is reversed and the levels of beta-amyloid and Tau protein in the brain tissue are restored to normal levels. Cognitive testing of these animals would show that the cognitive function is rescued including indications of Alzheimer's Disease.

Unless defined otherwise, all technical and scientific terms used herein have the same meanings as commonly understood by one of ordinary skill in the art to which the presently disclosed subject matter belongs. Although any methods, devices, and materials similar or equivalent to those described herein can be used in the practice or testing of the presently disclosed subject matter, representative methods, devices, and materials are now described.

### INDUSTRIAL APPLICABILITY

The present invention provides a method and composition for reversing cognitive impairments and loss using SGLT2 Inhibitors.

1. A method of treating cerebrovascular dementia which comprises administering to a subject in need thereof an effective amount of luseogliflozin or a pharmaceutically acceptable salt thereof.

2. The method according to claim 1 wherein the subject is an elderly subject.

3. The method according to claim 1 or 2, wherein the subject is a diabetic subject.

4. (canceled)
